(19) United States
(12) Patent Application Publication
(10) Pub. No.: US 2015/0112348 A1
(43) Pub. Date: Apr. 23, 2015

(54) MANIPULATE GUIDE REGISTRATION SURFACE

(71) Applicant: Biomet Manufacturing, LLC, Warsaw, IN (US)

(72) Inventors: Ryan J. Schoenefeld, Fort Wayne, IN (US); Kirk J. Bailey, Rochester, IN (US)

(21) Appl. No.: 14/518,224

(22) Filed: Oct. 20, 2014

Related U.S. Application Data
(60) Provisional application No. 61/893,562, filed on Oct. 21, 2013.

(51) Int. Cl. A61B 17/15; A61B 17/17

(52) U.S. Cl. CPC A61B 17/15; A61B 17/175; A61B 17/1764; A61B 17/1746; A61B 17/155
USPC 606/87

(57) ABSTRACT

A guide for an implant site in bone. The guide includes a bone mating surface configured to engage a prepared bone surface at the implant site to position the guide at a predetermined orientation, and a patient-specific surface configured to engage a predetermined portion of a specific patient's natural anatomy to position the guide at the predetermined orientation.

### CROSS-REFERENCE TO RELATED APPLICATIONS

This application claims benefit of U.S. Provisional Patent Application No. 61/893,562 filed on Oct. 21, 2013. The entire disclosure of the above application is incorporated herein by reference.

### FIELD

The present disclosure relates to a guide for directing instrumentation or an implant to an implant site.

### BACKGROUND

This section provides background information related to the present disclosure, which is not necessarily prior art.

Patient-specific alignment guides can be used as alternatives to standard orthopedic instrumentation and planning. Such guides are often based on complex imaging protocols from which three-dimensional models and preoperative plans are created. Exemplary imaging protocols include MRI, CT, x-ray, or ultrasound. Based on the preoperative plan, the patient-specific alignment guides are customized in order to position instrumentation at predetermined orientations to carry out the plan. The guides typically mate very closely and specifically with the bone, and often make use of bone surfaces and bony landmarks to insure proper orientation. The imaging is generally very accurate, which can be costly and time consuming. There is thus a need for a guide that can be precisely oriented based on less complex imagine modalities, which are less costly and less complex to prepare.

### SUMMARY

This section provides a general summary of the disclosure, and is not a comprehensive disclosure of its full scope or all of its features.

The present teachings provide for a guide for an implant site in bone. The guide includes a bone mating surface configured to engage a prepared bone surface at the implant site to position the guide at a predetermined orientation, and a patient-specific surface configured to engage a predetermined portion of a specific patient's natural anatomy to position the guide at the predetermined orientation.

The present teachings also provide for a guide for an implant site in bone. The guide includes a bone mating surface and a patient-specific surface. The bone mating surface is configured to engage a prepared bone surface at the implantation site to position the guide at a predetermined orientation. The patient-specific surface is configured to reference a natural bone surface of the patient to position the guide at the predetermined orientation, the patient-specific surface is a negative of the patient's natural bone surface such that the patient-specific surface nests with the patient's natural bone in only a single orientation. The guide is configured to direct devices to the implant site according to a preoperative plan.

The present teachings further provide for a method for guiding instrumentation to an implant site. The method includes: cutting bone at the implant site to define a reference surface on the bone configured to position a guide at a predetermined orientation relative to the bone when a bone mating surface of the guide is seated on the reference surface; mating the guide member with the reference surface to position the guide member at the predetermined orientation; and mating a patient-specific surface of the guide member with a natural, unmodified surface of the bone, the patient-specific surface configured to reference the natural, unmodified surface to position the guide at the predetermined orientation, the patient-specific surface is a negative of the natural, unmodified surface such that the patient-specific surface nests with the natural, unmodified surface in only a single orientation.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a guide according to the present teachings;

FIG. 2 is a side view of the guide of FIG. 1;

FIG. 3 is a perspective view of the guide of FIG. 1 positioned over an acetabulum prepared by reaming to receive an acetabular cup implant;

FIG. 4 illustrates the guide of FIG. 1 seated in the prepared acetabulum with an inserter;

FIG. 5 illustrates a guide pin guided to a hip bone by the guide;

FIG. 6 illustrates an acetabular cup implant implanted into the acetabulum with an implant inserter;

FIG. 7 illustrates a second guide pin seated in the hip bone and coupled to the first guide pin;

FIG. 8 is a side view of a femur cut with a suitable cutting instrument;

FIG. 9 illustrates an additional guide according to the present teachings, the guide mounted to the femur; and

FIG. 10 is a perspective view of the guide of FIG. 9 mounted to the femur.

Corresponding reference numerals indicate corresponding parts throughout the several views of the drawings.

### DETAILED DESCRIPTION

Example embodiments will now be described more fully with reference to the accompanying drawings.

The present teachings generally provide patient-specific surgical instruments that include, for example, alignment guides, drill guides, templates, cutting/resection guides for use in joint replacement, resurfacing procedures and other procedures related to the joint or the various bones of the joint, including adjacent bones. In various embodiments, the joint is a hip. In such embodiments, the present teachings generally provide a patient-specific acetabular guide or proximal femoral guide for use in orthopedic surgery, such as in joint replacement or revision surgery, for example. The patient-specific alignment guides can be used either with conventional or patient-specific implant components prepared with computer-assisted image methods.

In other embodiments, the joint is a knee. When the joint is a knee, the patient-specific surgical instruments can be used in knee joint replacement, resurfacing procedures and other procedures related to the knee joint or the various bones of the knee joint, including the femur and the tibia. The present teaching can be applied to partial and full knee reconstructions.

In a further embodiment, the joint is a shoulder. When the joint is a shoulder, the patient-specific surgical instruments can be used in shoulder joint replacement, resurfacing procedures and other procedures related to the shoulder joint or the various bones of the shoulder joint, including the glenoid and adjacent bones. The present teachings can be applied to anatomic shoulder replacement and reverse shoulder replacement.

The patient-specific instruments can be used either with conventional implant components or with patient-specific implant components and/or bone grafts that are prepared using computer-assisted image methods according to the present teachings. Computer modeling for obtaining two or three dimensional images of the patient's anatomy using MRI or CT, X-ray, or ultrasound scans of the patient's anatomy, the patient-specific prosthesis components and the patient-specific guides, templates and other instruments, can be designed using various CAD programs and/or software available, for example, by Materialise USA, of Plymouth, Mich.

The patient-specific instruments and any associated patient-specific implants and bone grafts can be generally designed and formed using computer modeling based on two or three dimensional anatomic image(s) generated from X-rays, MRI, CT, ultrasound or other medical scans. Specifically, an anatomical feature (e.g., a scapula, knee, or pelvis) can be imaged to detect certain features of the anatomy (e.g., dimensions, curvature of surfaces, etc.). Then, patient-specific instruments can be formed according to these measurements. Various pre-operative procedures are disclosed in commonly assigned U.S. Pat. No. 8,092,465, issued on Jan. 10, 2012, U.S. patent Publication No. 2011/0184419, published on Jul. 28, 2011, and U.S. Publication No. 2012/0310399, published on Dec. 6, 2012, which are all incorporated herein by reference in their entirety.

In some embodiments, the patient-specific instrument can have a three-dimensional engagement surface that is a mirror image or negative of a boney surface or cartilage. The three-dimensional engagement surface is complementary to and made to conformingly contact, engage, or nest on a bony anatomical surface or cartilage. Thus, the patient-specific instruments can be configured to fit at only one position to the anatomical surface. The patient-specific instruments can include custom-made guiding formations, such as, for example, guiding bores or cannulated guiding posts or cannulated guiding extensions or receptacles that can be used for supporting or guiding other objects, such as instruments, drill guides, reamers, cutters, cutting guides and cutting blocks or for inserting pins or other fasteners according to a surgeon-approved pre-operative plan.

In various embodiments, the patient-specific instruments can also include one or more patient-specific alignment guides for receiving and guiding a tool, such as a drill or pin or guide wire at corresponding patient-specific orientations relative to a selected anatomic axis for the specific patient. The patient-specific instruments can include guiding or orientation formations and features for guiding the implantation of patient-specific or off-the-shelf implants associated with the surgical procedure. The geometry, shape and orientation of the various features of the patient-specific instruments, as well as various patient-specific implants and bone grafts, if used, can be determined during the pre-operative planning stage of the procedure in connection with the computer-assisted modeling of the patient's anatomy. During the pre-operative planning stage, patient-specific instruments, custom, semi-custom or non-custom implants and other non-custom tools, can be selected and the patient-specific components can be manufactured for a specific-patient with input from a surgeon or other professional associated with the surgical procedure.

In the following discussion, the terms “patient-specific”, “custom-made” or “customized” are defined to apply to components, including tools, implants, portions or combinations thereof, which include certain geometric features, including surfaces, curves, or other lines, and which are made to closely conform as mirror-images or negatives or complementary surfaces of corresponding geometric features or anatomic landmarks of a patient's anatomy obtained or gathered during a pre-operative planning stage based on two or three dimensional computer images of the corresponding anatomy reconstructed from image scans of the patient by computer imaging or X-ray methods. Further, patient-specific guiding features, such as, guiding apertures, guiding slots, guiding members or other holes, openings, or guide surfaces that are included in alignment guides, drill guides, cutting guides, rasps or other instruments or in implants are defined as features that are made to have positions, orientations, dimensions, shapes and/or define cutting planes and axes specific to the particular patient's anatomy including various anatomic or mechanical axes based on the computer-assisted pre-operative plan associated with the patient.

The prepared patient-specific alignment guides can be configured to mate in alignment natural boney anatomic landmarks by orienting and placing the corresponding alignment guide intra-operatively on top of the bone to mate with corresponding boney landmarks. The boney landmarks function as passive fiducial identifiers or fiducial markers for positioning of the various alignment guides, drill guides or other patient-specific instruments.

The various patient-specific alignment guides can be made of any biocompatible material, including, polymer, ceramic, metal or combinations thereof. The patient-specific alignment guides can be opaque, semi-transparent, or transparent. The patient-specific alignment guides can be disposable and can be combined or used with reusable and non patient-specific cutting and guiding components.

More specifically, the present teachings provide various embodiments of patient-specific acetabular, knee, glenoid, or other appropriate guides. The acetabular, knee, glenoid or other appropriate guides of the present teachings can have patient-specific engagement surfaces that reference various portions of the hip, knee, or shoulder joint and include drill guides, guiding bores or sleeves or other guiding formations that can accurately position a guide wire for later acetabular, knee, or glenoid preparation and implantation procedures and for alignment purposes, including implant position control, implant version control, implant inclination control.

In the following, when of portion of a patient-specific guide is described as “referencing” a portion of the anatomy, it will be understood that the referencing portion of the patient-specific guide is a patient-specific portion or surface mirroring or negative to the corresponding referenced cartilage surface and/or bone surface. Exemplary, non-limiting patient-specific guides are shown, but additional patient-specific guides can be configured based on the present teachings.

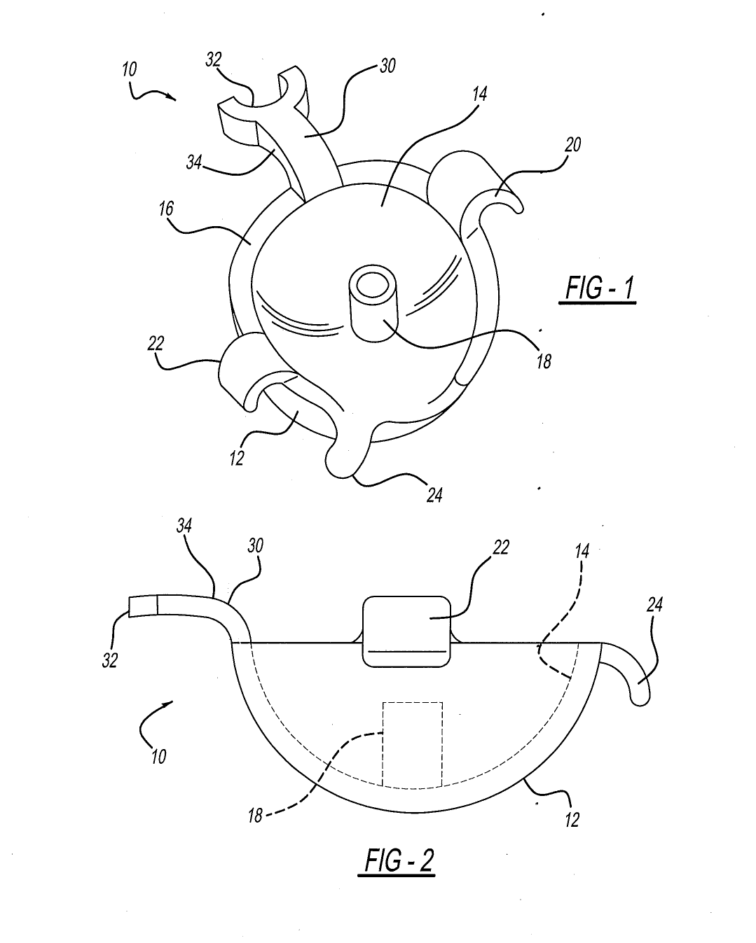

With initial reference to FIGS. 1 and 2, a guide according to the present teachings is generally illustrated at reference numeral 10. The guide 10 includes an outer or bone mating surface 12 and an inner surface 14 opposite thereto. The bone mating surface 12 is generally convex and spherical. The inner surface 14 is generally concave. The guide 10 is an acetabular cup guide, but can be of any suitable size and shape corresponding to any suitable implant site and bone. Extending between the bone mating surface 12 and the inner surface 14 is a rim 16.

The bone mating surface 12 is sized and shaped to generally correspond to a non-patient specific bone surface that has been prepared to receive an implant. For example and as described herein, the bone mating surface 12 can be sized and shaped to generally correspond to a reamed surface of an acetabulum. The non-patient specific bone surface is not customized to a particular patient or natural bone surface.

The guide 10 further includes an inserter coupling member 18. The inserter coupling member 18 is illustrated as a flange protruding from the inner surface 14 generally at an axial center thereof. However, the inserter coupling member 18 can be of any suitable shape or size to couple with a suitable insertion device for inserting the guide 10 at an implantation site, as further described herein.

The guide 10 includes a first patient-specific member 20, a second patient-specific member 22, and a third patient-specific member 24. As illustrated, the first and second patient-specific members 20 and 22 extend from and above the rim 16, and generally extend from opposite surfaces thereof. The first and second patient-specific members 20 and 22 are illustrated as flanges, but can be any suitable member or device configured to engage specific portions of the patient's anatomy to orient the guide 10 at a predetermined orientation pursuant to a preoperative plan. The third patient-specific member 24 can extend from the rim 16 or any suitable portion of the guide 10. The patient-specific member 24 can extend below the rim 16 as illustrated, or in any other suitable direction. The third patient-specific member 24 can be similar to the first and second patient-specific members 20 and 22, and thus configured to engage a specific portion of the patient's anatomy in order to orient the guide 10 in a desired rotational orientation.

The first, second, and third patient-specific members 20, 22, and 24 can be any suitable patient-specific members configured to mate and/or nest with a particular surface or portion of a specific patient's natural bone in only a single orientation. For example, the first, second, and third patient-specific members 20, 22, and 24 can be configured to have a negative shape of the patient's unaltered bone or a non-modified bone surface.

The guide 10 also includes a guide pin locator 30. The guide pin locator 30 includes a generally U-shaped flange 32 mounted to the rim 16 with a support arm 34. The U-shaped flange 32 can have any suitable size or shape, such as other than U-shaped, to permit cooperation with or referencing off of a guide pin, as further described herein. Although the guide pin locator 30 is illustrated as extending from the rim 16, the guide pin locator 30 can be at any suitable location of the guide 10.

The first, second, and third patient-specific members 20, 22, and 24 are generally illustrated as flanges, but can be any suitable size or shape to reference off of bone surfaces of a specific patient. The first, second, and third patient-specific members 20, 22, and 24 are generally illustrated as flanges extending from the rim 16. However, each one of the first, second, and third patient-specific members 20, 22, and 24 can be at any suitable location of the guide 10, and can take the form of any suitable size or shape, in order to reference predetermined surface features of the patient's native bone. For example, the first, second, and third patient-specific members 20, 22, and 24 can be any suitable patient-specific members configured to mate and/or nest with a particular surface or portion of a specific patient's natural bone in only a single orientation. These native bone features can be, for example, protruding bone ridges or depressions in bone. Any suitable number of the first, second, and third patient-specific members 20, 22, and 24 can be provided, and all need not be included. For example, the first, second, and third patient-specific members 20, 22, and 24 can take the form of a single flange that extends from at least a portion of the rim 16 outward therefrom in order to engage, for example, a bony ridge or rim at an outer periphery of an acetabulum.

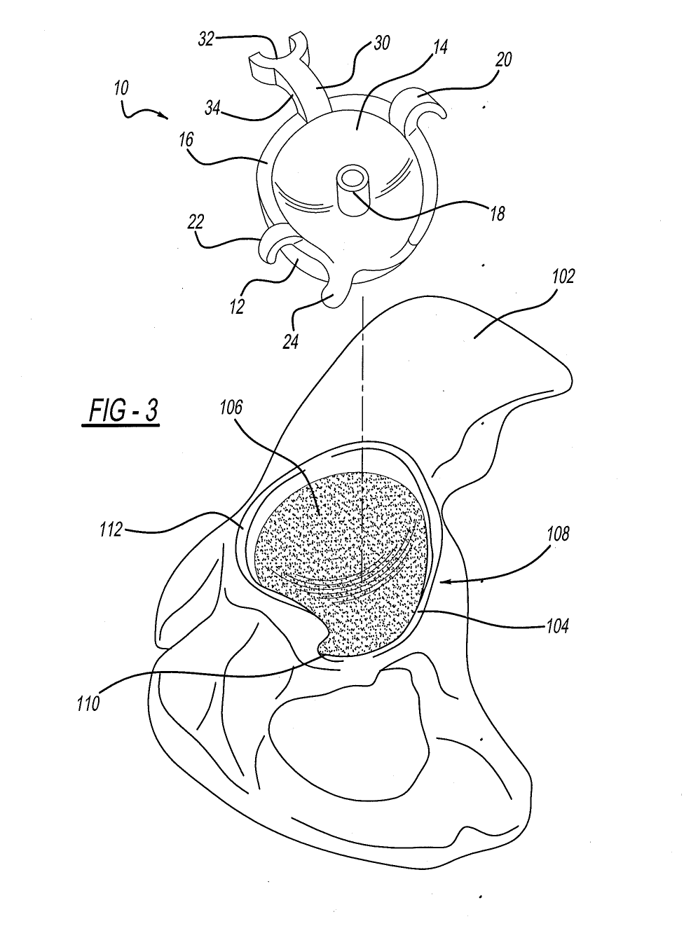

The location, size, shape, and number of the first, second, and third patient-specific members 20, 22, and 24 can be determined based on imaging of a specific patient. Any suitable form of imaging of the patient's acetabulum can be performed, such as with a CT scan or x-ray. The imaging techniques need not include cartilage, which can often simplify imaging of the patient's bone. By referencing specific features of the patient's bone, 10 guide 10 can be implanted in a custom orientation to guide additional instrumentation to the patient's bone depending on the patient's specific anatomy. For example, the guide 10 can guide an acetabular cup implant to the patient's acetabulum as illustrated in FIGS. 3-7, which will now be described in detail.

FIG. 3 illustrates a hip bone of a specific patient at reference numeral 102. An acetabulum of the hip bone 102 is illustrated at reference numeral 104. The acetabulum 104 includes a reference surface or prepared bone surface 106 therein. The prepared bone surface 106 is prepared using any suitable instrument, such as a reamer, in order to prepare the acetabulum 104 to receive an implant. The acetabulum 104, the prepared bone surface 106 thereof, and portions of the hip bone 102 surrounding the acetabulum 104 together generally define an implant site 108. The implant site 108 includes various patient-specific features, such as bony recess 110 and an outer periphery 112 of the acetabulum 104. The implant site 108 also includes the prepared bone surface 106, which is non-patient specific and can be a reamed surface, for example.

The prepared bone surface 106 can be of any suitable size and shape based on the patient's anatomy and the implant. The size and shape of the guide 10, such as the bone mating surface 12 and the first, second, and third patient-specific members 20, 22, and 24, can be of any suitable size or shape to correspond to the implant site 108 such that the implant site 108 receives the guide 10 in only a single patient-specific orientation pursuant to the preoperative plan in order to guide an implant to the acetabulum for implantation at a predetermined orientation.

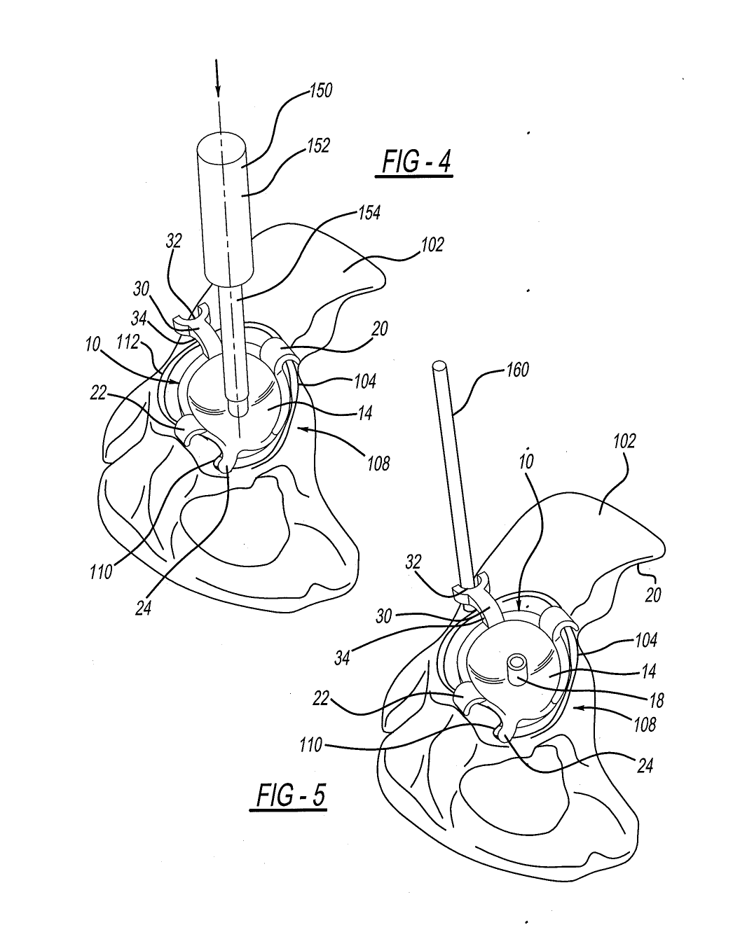

With reference to FIG. 4, a guide inserter for inserting the guide 10 in the acetabulum 104 is illustrated at reference numeral 150. The guide inserter 150 can be any suitable insertion device capable of coupling with the guide 10. For example, the guide inserter 150 includes a handle 152 with a shaft 154 extending therefrom. The shaft 154 is configured to couple with the inserter coupling member 18 in order to connect the guide inserter 150 to the guide 10. The guide inserter 150 can be used to position the guide 10 within the acetabulum 104 such that the bone mating surface 12 generally mates with, or abuts, the prepared bone surface 106, and the first, second, and third patient-specific members 20, 22, and 24 reference various predetermined patient-specific bone portions at the implant site 108. For example, and as illustrated in FIG. 4, the third patient-specific member 24 can cooperate with the bony recess 110, and the first and second patient-specific members 20 and 22 can reference off of the periphery 112 of the acetabulum 104, in order to position the guide 10 at a predetermined orientation for guiding an implant to the acetabulum 104. By referencing various predetermined patient-specific bone portions at the implant site 108, the first, second, and third patient-specific members 20, 22, and 24 allow the guide 10 to nest in only one orientation, which corresponds to an orientation identified during preparation of the pre-operative plan.

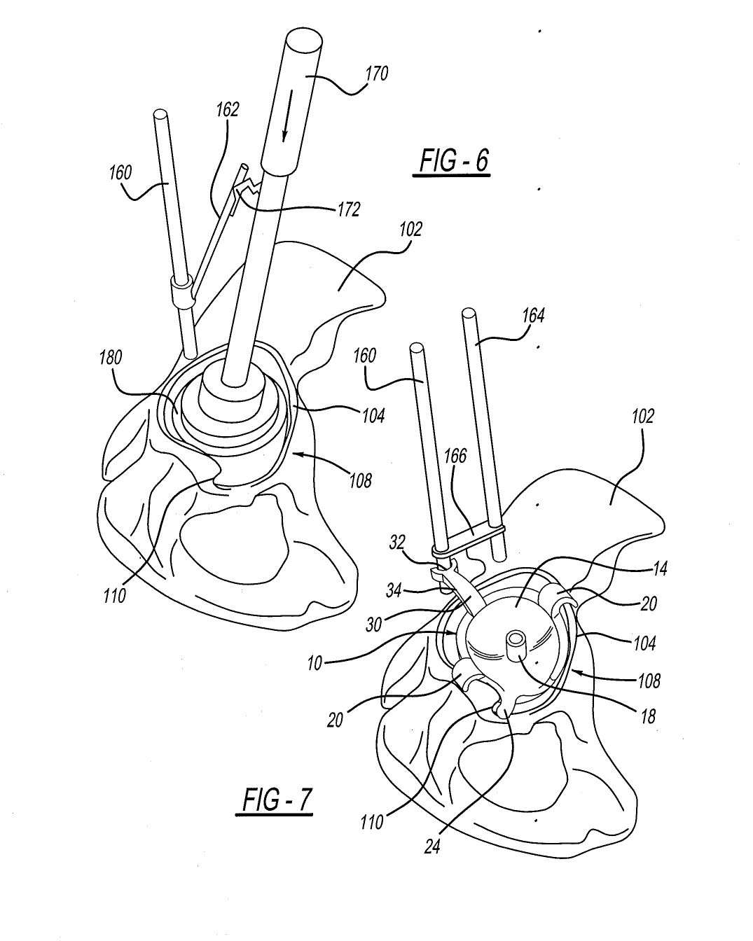

The guide pin locator 30 is arranged to guide a referencing member, such as a first guide pin 160 of FIG. 4, to the hip bone 102. The guide pin locator 30 is positioned in order to direct the first guide pin 160 to the hip bone 102 at a predetermined position and orientation to fit the patient's anatomy. After the first guide pin 160 is inserted in the hip bone 102 into a position guided by the guide pin locator 30, the guide 10 is removed from the acetabulum 104. With reference to FIG. 6, a reference arm 162 is coupled to the first guide pin 160. Further description of reference arms similar to reference arm 162 can be found in, for example, U.S. Publication No. 2011/0184419 (published on Jul. 28, 2011), which was incorporated by reference above.

An implant inserter 170 is coupled to acetabular cup implant 180 in any suitable manner in order to implant the acetabular cup implant 180 within the acetabulum 104. The implant inserter 170 includes a reference flange 172 for referencing off of the reference arm 162. The implant inserter 170 can be any suitable insertion device for the acetabular cup implant 180, such as an impactor. The acetabular cup implant 180 can be any suitable acetabular cup implant, such as the various acetabular cup implants provided by Biomet of Warsaw, Ind.

With the acetabular cup implant 180 connected to the implant inserter 170, the implant inserter 170 is positioned such that the reference flange 172 thereof is generally aligned with, or in cooperation with, the reference arm 162. With the acetabular cup implant 180 positioned at the implant site 108, the acetabular cup implant 180 is impacted into the acetabulum 104 using the implant inserter 170.

Due to variations in patient anatomy, the patient's hip bone 102 may not have sufficient rigidity or structure where the first guide pin 160 is to be mounted. In such cases, a second guide pin can be used. The second guide pin 164 can be inserted into the hip bone 102 at any location where there is sufficient bone rigidity, and coupled to the first guide pin 160 with a suitable coupling member, such as the bridge 166. The first guide pin 160 can thus be supported at an area where there is insufficient structure of the hip bone 102 by the second guide pin 164 by coupling the first guide pin 160 to the second guide pin 164 with the bridge 166.

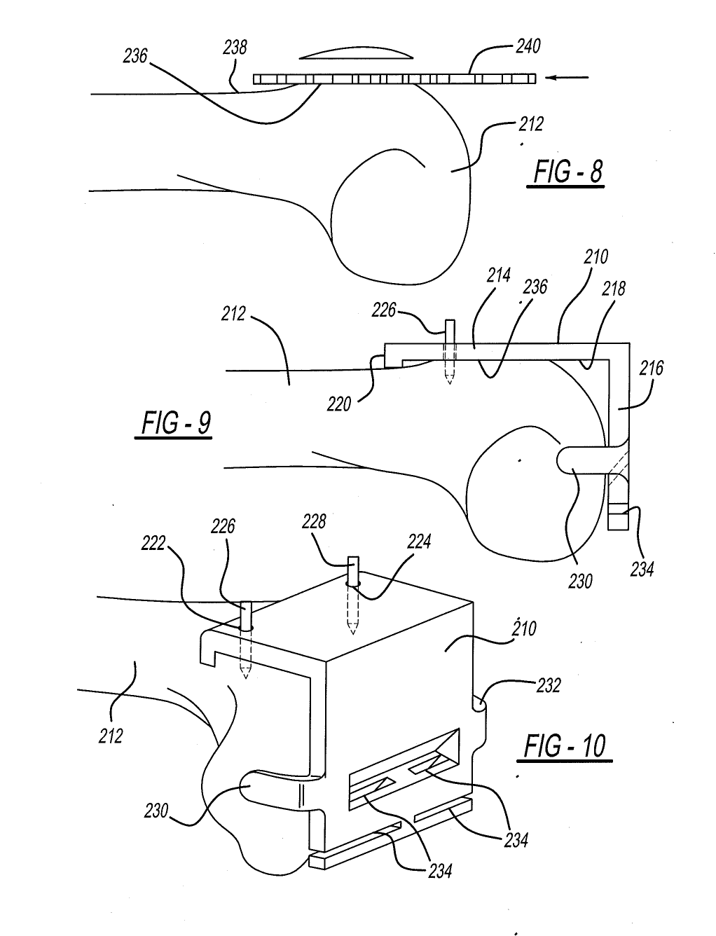

With reference to FIGS. 8-10, another guide according to the present teachings is generally illustrated at reference numeral 210. The guide 210 is generally illustrated as a cutting guide for bone, such as the femur 212. The guide 210 generally includes an anterior portion 214 and an inferior portion 216. The anterior portion 214 and the inferior portion 216 can be provided at any suitable orientation relative to one another, such as generally at a right angle as illustrated. The guide 210 further includes a bone mating surface 218, which extends along an interior of each of the anterior portion 214 and the inferior portion 216. The bone mating surface 218 is generally planar, such as at the anterior portion 214. At an end of the anterior portion 214 opposite to the inferior portion 216 is a flange 220. The anterior portion 214 defines a first aperture 222 and a second aperture 224 extending therethrough, each of which are sized and shaped to receive a first retention pin 226 and a second retention pin 228 respectively. The first and second retention pins 226 and 228 can be used to secure the guide 210 to the femur 212, or any other suitable bone surface.

The inferior portion 216 of the guide 210 includes the first patient-specific member 230 and a second patient-specific member 232. The first and second patient-specific members 230 and 232 can be any suitable portion of the guide 210, or members coupled to the guide 210, which are configured to mate and/or nest with a particular surface or portion of a specific patient's natural bone in only a single orientation. The first and second patient-specific members 230 and 232 can be configured to have a negative shape of the patient's unaltered bone or a non-modified bone surface. For example and as illustrated, the first and second patient-specific members 230 and 232 are flanges sized and shaped to reference off of opposing lateral surfaces of the femur 212. By referencing off of patient-specific surfaces of the femur 212 the first and second patient-specific members 230 and 232 can orient the guide 210 at a predetermined orientation, such as to guide a suitable cutting instrument to the femur 212 through cutting slots 234 of the guide 210.

At the anterior portion 214, the bone mating surface 218 references off a reference surface or prepared bone surface 236 at an anterior surface 238 of the femur 212. The prepared bone surface 236 is generally planar, and can be prepared using any suitable cutting instrument, such as cutting blade 240. By referencing off of the prepared bone surface 236, the guide 210 can be supported at a suitable orientation for directing a cutting instrument to the femur 212 through the cutting slots 234 according to a preoperative surgical plan in order to prepare the femur 212 to receive an implant. Because the guide 210 references off of the prepared bone surface 236 of a known shape and orientation, less sophisticated bone imaging modalities can be used, such as x-ray imaging, to prepare the pre-operative plan and determine proper orientation of the guide 210.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A guide for an implant site in bone comprising:
a bone mating surface configured to engage a prepared bone surface at the implant site to position the guide at a predetermined orientation; and
a patient-specific surface configured to engage a predetermined portion of a specific patient's natural anatomy to position the guide at the predetermined orientation.

2. The guide of claim 1, wherein the guide is an acetabular cup guide.

3. The guide of claim 1, wherein the guide is a cutting guide.

4. The guide of claim 3, wherein the guide is a femoral cutting guide.

5. The guide of claim 1, wherein the bone mating surface is spherical.

6. The guide of claim 1, wherein the bone mating surface is concave.

7. The guide of claim 1, wherein the bone mating surface is configured to mate with a reamed acetabulum.

8. The guide of claim 1, wherein the bone mating surface is planar.

9. The guide of claim 1, wherein the bone mating surface is configured to mate with a planar cut surface of a femur.

10. The guide of claim 1, wherein the patient-specific surface nests with the patient's bone surface at only one orientation.

11. The bone preparation guide of claim 10, wherein the patient-specific surface is a flange configured to engage a bone surface proximate to the patient's acetabulum.

12. The bone preparation guide of claim 10, wherein the patient-specific surface is a flange configured to engage a surface of the patient's femur.

13. A guide for an implant site in bone comprising:
a bone mating surface configured to engage a prepared bone surface at the implantation site to position the guide at a predetermined orientation; and
a patient-specific surface configured to reference a natural bone surface of the patient to position the guide at the predetermined orientation, the patient-specific surface is a negative of the patient's natural bone surface such that the patient-specific surface nests with the patient's natural bone in only a single orientation;
wherein the guide is configured to direct devices to the implant site according to a preoperative plan.

14. The guide of claim 13, wherein the guide is one of an acetabular cup guide or a femoral cut guide.

15. The guide of claim 13, further comprising a guide surface including at least one of a hole, edge, or slot.

16. The guide of claim 13, wherein the devices include one of an acetabular cup implant or a bone cutting instrument.

17. The guide of claim 13, wherein the bone mating surface is one of a planar surface or a convex spherical surface configured to mate with an acetabulum.

18. A method for guiding instrumentation to an implant site comprising:
cutting bone at the implant site to define a reference surface on the bone configured to position a guide at a predetermined orientation relative to the bone when a bone mating surface of the guide is seated on the reference surface;
mating the guide member with the reference surface to position the guide member at the predetermined orientation; and
mating a patient-specific surface of the guide member with a natural, unmodified surface of the bone, the a patient-specific surface configured to reference the natural, unmodified surface to position the guide at the predetermined orientation, the patient-specific surface is a negative of the natural, unmodified surface such that the patient-specific surface nests with the natural, unmodified surface in only a single orientation.

19. The method of claim 18, wherein the predetermined orientation is determined based on an imaging modality that is devoid of soft tissue or cartilage.

20. The method of claim 18, wherein cutting bone includes one of reaming an acetabulum or resecting the femur.

21. The method of claim 18, further comprising guiding the instrumentation to the implant site along a guide surface and positioning one of a final implant or a resection device using the guide surface.
